(19) United States
(12) Patent Application Publication
(10) Pub. No.: US 2026/0240099 A1
(43) Pub. Date: Aug. 20, 2026

(54) METHODS OF PRODUCING CHOCOLATE FROM CACAO PLANT CELL MATERIALS

(71) Applicant: CALIFORNIA CULTURED INC, Davis, CA (US)

(72) Inventors: Steven William STEARNS, Sacramento, CA (US); Rio Angelo STAMLER, Davis, CA (US); Michael VILLANUEVA, Davis, CA (US); Alan PERLSTEIN, Davis, CA (US)

(73) Assignee: CALIFORNIA CULTURED INC, Davis, CA (US)

(21) Appl. No.: 19/326,716

(22) Filed: Sep. 11, 2025

Related U.S. Application Data
(63) Continuation of application No. PCT/US2025/016531, filed on Feb. 20, 2025.

(51) Int. Cl. A01H 4/00; A23G 1/00; A23G 1/48

(52) U.S. Cl. CPC A01H 4/002; A23G 1/0006; A23G 1/0016; A23G 1/0036; A23G 1/48

(57) ABSTRACT

The present invention relates to methods of producing chocolate, chocolate-like or food products, or food materials, from plant cell culture somatic embryogenesis biomass. The methods include culturing an in vitro cacao plant cell biomass, optionally with at least one microorganism and/or at least one enzyme, wherein the biomass comprises somatic embryos (SEs) from Theobroma cacao L. explant material. Chocolate, chocolate-like and food products obtained from such methods are also provided.

### CROSS-REFERENCES TO RELATED APPLICATIONS

This application claims the benefit of International Application PCT/US2025/016531 filed on Feb. 20, 2025, which itself claims the benefit of U.S. Provisional Patent Application No. 63/556,187 filed on Feb. 21, 2024; both applications are incorporated herein by reference in their entirety.

### FIELD OF THE INVENTION

The present invention relates to methods of producing chocolate, chocolate-like or food products, or food materials, from plant cell culture somatic embryogenesis biomass. The methods include culturing an in vitro cacao plant cell biomass, optionally with at least one microorganism and/or at least one enzyme, wherein the biomass comprises somatic embryos (SEs) from Theobroma cacao L. explant material. Chocolate, chocolate-like and food products obtained from such methods are also provided.

### BACKGROUND OF THE INVENTION

Chocolate production faces several challenges, including economic, environmental, and social issues. Some of the problems associated with chocolate production include child labor and exploitation, deforestation, use of pesticides and fertilizers in cacao cultivation, climate change, crop disease, and significant water usage. Cacao farm productivity is impacted by these issues.

Propagation of cacao plants has been traditionally carried out using rooted cuttings or by grafting, but these techniques require time and labor investments to obtain material with the desired genetic traits and health. In the search for solutions to this problem, plant cell culture has been investigated for the propagation and regeneration of Theobroma cacao L. cacao plants. Somatic embryogenesis (SE) is one of the techniques employed for the clonal propagation of cacao plants. It involves collecting somatic (non-reproductive) explant materials, such as staminodes or epicotyls, and placing them in culture medium to form somatic embryos. With the right combination of nutrients and growth regulators, these tissues can develop into plantlets in vitro.

As explained in Garcia C, et al., “Somatic Embryogenesis in Theobroma cacao L.”, Methods Mol Biol. (2018); 1815:227-245, SE is a vegetative method used for cacao propagation that offers significant technological advantages over conventional methods, including the production of a large number of plants from a small amount of tissue, based on elite cacao varieties, as well as genetic improvement by enabling the selection and regeneration of plants with desirable traits, such as disease resistance or improved yield. GB 2537153A describes how somatic embryogenesis in plants is useful for the regeneration of genetically uniform plant material. EP 0293598 A2, WO 2023/076272 A1, US 2001/0047524 A1, U.S. Pat. Nos. 4,545,147, 4,301,619 A and US 2022/0232790 A1 disclose the use of in vitro plant cell culture methods, specifically somatic embryogenesis/asexual embryogenesis, to produce and propagate Theobroma cacao plants. However, there are significant risks of producing mutants with the technique. In particular, methods involving chemical induction carry a relatively high risk of genetic or epigenetic mutations, so called somaclonal variation. Therefore, maintaining the genetic stability of the material is one important aspect of the plant propagation methods using SE.

In particular, U.S. Pat. No. 10,701,876 B2 describes methods for propagating Theobroma cacao L. plants in vitro, including the multiplication of somatic embryos with exposure to light using direct or indirect somatic embryogenesis, to regenerate cacao plants. U.S. Pat. No. 8,921,087 B2 describes in vitro multiplication and plant regeneration of Theobroma cacao L. to achieve clonal propagation by SE. The methods described in these documents are purposely limited to two steps of multiplication and development (i.e., secondary somatic embryos) to limit the risks of somaclonal variation in the embryos produced.

Bustami Mirni Ulfa, et al., “Cyclic Somatic Embryogenesis in Indonesian Elite Theobroma cacao L. Clones”, HORTICULTURAE, vol. 10, no. 1, 25 Dec. 2023 (2023-12-25), page 24, explores advancements and interests in the generation and propagation of cocoa plants, with a focus on in vitro SE as a promising method for large-scale plant propagation. Successive cycles of SE are presented as advantageous because they are self-sustaining and do not require repeated use of the original explant. However, the authors also acknowledge the risks of somaclonal variation in prolonged culture due to genetic and epigenetic variation, which is key to plant propagation. Cyclic secondary somatic embryogenesis is presented as promising for the mass production of clones. The prolific and synchronized development of secondary embryos allows for a more consistent and scalable supply of embryonic material. This is particularly advantageous in genetic engineering and GMO studies, where researchers often need many embryos to test and validate genetic modifications.

Plant cell culture has also been employed for the production of secondary metabolites, such as plant polyphenols, which can be used as pharmaceuticals, food additives and flavors.

Plants inherently synthesize a diverse array of metabolites, serving as essential products and intermediates in their metabolic processes. These metabolites play multifaceted roles, encompassing vital functions such as signaling, enzyme inhibition, defense mechanisms, coenzyme facilitation, and safeguarding against a spectrum of pathogens and diseases. Theobroma cacao has been recognized to produce both primary and secondary metabolites of different types. Plants engage in the processes of photosynthesis and respiration, utilizing carbon dioxide as the exclusive source of carbon, along with water, solar energy, and ammonia as initial substrates. These biochemical pathways orchestrate the synthesis of essential primary metabolites, including but not limited to glucose, amino acids, lipids, and nucleic acids, thereby sustaining the intricate molecular machinery fundamental to their growth, development, and physiological functions. Plants additionally excrete biologically active chemical compounds that do not directly participate in the developmental and growth processes of plants. These compounds, categorized as secondary metabolites, serve distinct roles beyond the foundational life processes, contributing to a spectrum of ecological interactions, defense mechanisms, and adaptive responses within the plant kingdom. Examples of secondary metabolites include alkaloids, flavonoids, anthocyanins (e.g., procyanidins), saponins, tannins, phenols, glycosides, terpenoids, coumarins, lignans, carotenoids, tocopherols and tocotrienols, sterols, and glucosinolates. Some of these secondary metabolites have been identified for their pharmacological/toxicological response in humans while increasing healthy nutrition as the major component of nutraceuticals and dietary supplements. They can also help increase agricultural yields by acting as growth enhancers of plants and animals as well as being used as dyes, fragrances, flavors, and cosmetics.

Pence, V. C., et al., “Sucrose-mediated regulation of fatty acid composition in asexual embryos of Theobroma cacao”, Physiol. Plant. (1981) 53, 378-384, studied the regulation of secondary metabolites in plant cell and tissue culture. The article additionally describes that fatty acid (FA) biosynthesis in somatic embryos of Theobroma cacao L. can be regulated in vitro similar to the one which occurs in vivo in the zygotic embryo during normal maturation.

U.S. Pat. No. 9,167,840 B2 relates to a method of preparing cacao oligomeric procyanidins, the method comprising: culturing cacao callus cells derived from a Theobroma cacao plant in a culture medium for a time sufficient and under conditions sufficient to result in production of cacao oligomeric procyanidins at a first rate; and inducing the cells to produce the cacao oligomeric procyanidins at a second rate that is higher than the first rate by introducing a selected amount of a carbohydrate to the cells, wherein the step of culturing the cacao cells is carried out in the presence of dissolved oxygen concentration at 1% to 400% of air saturation.

As described in Eibl, et al., “Plant cell culture technology in the cosmetics and food industries: current state and future trends”, Applied Microbiology and Biotechnology (2018) 102:8661-8675 and patent application EP 4176710 A1, stress response elicitation, i.e., exposing plants to specific stimuli and conditions that challenge their normal growth and development, is widely applied to increase the production of plant cell culture-based secondary metabolites.

Plant cell culture may also be employed to manufacture cocoa powder from cacao plant cell cultures in suspensions, as described in U.S. Pat. No. 4,306,022 A, as well as in Eibl, et al. (2018) and EP 4176710 A1. Because the method described by Eibl, et al. does not involve post-harvest bean processing, including fermentation, and often alkalinization, which destroyed significant amount of polyphenols, the cocoa powder obtained from such cell cultures is presented as advantageously exhibiting a lower acetic acid content, which is considered as leading to unpleasantly sour, pungent and bitter flavors in the final cocoa products and tending to mask desirable aromas perceived as pleasant. The methods involve culturing cells obtained from callus from cacao bean explant or from cacao beans treated with a cell wall disaggregating enzyme in a bioreactor selected among a stirred tank bioreactor, an orbitally shaken bioreactor, a bubble column bioreactor or an air-lift bioreactor. More precisely, the methods involve inducing callus from a cacao bean, propagating the callus, selecting a callus clone, initiating a suspension cell culture, and growing the suspension cells to produce a biomass comprising secondary metabolites selected from polyphenols, vitamins and aromatic compounds. The biomass is then separated from the suspension cell culture to provide a cocoa powder. Because the biomass does not produce enough lipids, the extract is mixed with cocoa butter (i.e., an essential component of chocolate) in a significant amount to produce chocolate. This comes with major economic and production drawbacks. First, high-quality cocoa butter is expensive and raises potential issues of sourcing. Additionally, the processing steps needed to formulate the final product add to the overall production cost.

The systems described in the state of the art so far do not provide methods which can be implemented at an industrial scale and lead to products that are comparable to cacao bean products.

In contrast, the method of the present invention is directed to producing chocolate, chocolate-like products, food products, or food materials, from an in vitro cacao plant cell biomass, wherein the biomass comprises somatic embryos (SEs) from Theobroma cacao L. explant material.

### BRIEF STATEMENT OF THE INVENTION

The methods of the invention allow the production of chocolate, chocolate-like or food products based on cacao cell lines generated through somatic embryogenesis.

The present invention provides a method of producing chocolate, chocolate-like products, food products or food materials from plant cell culture somatic embryogenesis biomass, comprising the steps of:
a) culturing an in vitro cacao plant cell biomass in a cell culture medium, optionally with at least one microorganism and/or at least one enzyme, wherein the biomass comprises somatic embryos from Theobroma cacao L. explant material;
b) separating the plant cell biomass from the cell culture medium;
c) optionally drying the biomass from step b) at least partially to obtain an at least partially dried biomass;
d) optionally roasting the biomass from step b) or at least partially dried biomass from step c) to obtain a roasted biomass;
e) optionally grinding the biomass from step b), the at least partially dried biomass from step c) or the roasted biomass from step d) to obtain a ground material;
f) optionally conching the ground material from step e) to obtain a conched material; and
g) optionally tempering the ground material from step e) or the conched material from step f).

The present invention provides chocolate, chocolate-like products, food products or food materials comprising somatic embryos (SEs) from Theobroma cacao L. explant material, which can be obtained from such methods.

### BRIEF DESCRIPTION OF THE DRAWINGS

FIG. 1 schematically illustrates an exemplary method of producing in vitro lipid-rich cacao plant cell biomass which is used in step a) of the method of the present invention.

FIG. 2 schematically illustrates another exemplary method of producing in vitro lipid-rich cacao plant cell biomass which is used in step a) of the method of the present invention.

FIG. 3 is a photograph of matured SE material after 24-hour drying, having a high lipid content, which has been used to prepare chocolate (example 7).

FIG. 4 is a photograph of a cell suspension after 5 months in culture containing no somatic embryos (Comparative—See example 5).

### DETAILED DESCRIPTION OF THE INVENTION

In the present application:
the expression “comprised between . . . and . . . ” should be understood as including the limits;
any description, even though described in relation to a specific embodiment, is applicable to and interchangeable with other embodiments of the present invention;
where an element or component is said to be included in and/or selected from a list of recited elements or components, it should be understood that in related embodiments explicitly contemplated here, the element or component can also be any one of the individual recited elements or components, or can also be selected from a group consisting of any two or more of the explicitly listed elements or components; any element or component recited in a list of elements or components may be omitted from such list; and
any recitation herein of numerical ranges by endpoints includes all numbers subsumed within the recited ranges as well as the endpoints of the range and equivalents.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

The present invention generally relates to methods of producing (or methods of manufacturing, equivalently used herein) chocolate, chocolate-like products, food products or food materials, based on in vitro cacao plant cell biomass, more precisely from plant cell culture somatic embryogenesis biomass.

While regulations typically impose specific compositional requirements for both chocolate and chocolate-like products, chocolate products are generally defined as being derived from cocoa beans. In contrast, chocolate-like products imitate the taste, texture, and appearance of chocolate but differ in their composition, such as in their fat or sugar content. The present invention is not limited to any particular food products.

In the context of the present invention, the term “food material” refers to any ingredient or component used in the formulation of chocolate, chocolate-like product, or food product, including but not limited to raw materials, intermediates, and additives that contribute to the product's final composition, functionality, or sensory attributes. The term “food additive” refers to any substance intentionally incorporated into chocolate, chocolate-like product, or food product, to enhance its properties, such as flavor, texture, stability, or nutritional value. The term “food material” therefore includes food additives. For sake of clarity, the plant cell biomass or SEs biomass obtained from the methods of the invention, wherein such biomass has optionally been cultured with at least one microorganism and/or at least one enzyme, and wherein the biomass comprises somatic embryos (SEs) from Theobroma cacao L. explant material, do qualify herein as food material.

The present invention is directed to methods of producing chocolate, chocolate-like products, food products or food materials from plant cell culture somatic embryogenesis. Key elements of the methods of the invention are as follows. The plant cell materials, including the SEs biomass and any valuable biomass extract, akin to the SEs biomass, are used to produce chocolate, chocolate-like products, food products or food materials. The SEs biomass may be combined with additional components (for example carbohydrates). The SEs biomass may be optionally subjected to fermentation with the addition of yeast, bacteria or enzymes in liquid or solid media. The SEs biomass may also undergo acidic incubation for a specific duration. The SEs biomass can be heat-roasted to facilitate the accumulation of compounds associated with the Maillard reaction. The SEs biomass may be subjected sequentially to at least one of fermentation, incubation, roasting, conching, and grinding, to produce the chocolate, chocolate-like products, food products or food materials. Either the SEs biomass or its extract can undergo conching for a determined duration to modify the aroma profile within the resulting chocolate. In the methods and products described herein, the added components may constitute between about 0.1 wt. % to about 80 wt. % of the finished product, for example between about 5 and about 50 wt. % or between about 10 and about 40 wt. %.

Cacao cell biomass (or extract) replaces cocoa butter, at least in part, in the methods, products and materials of the invention, which provides multiple advantages. One of them is that producing cacao cell lines which are suited to replace cacao butter reduces or eliminates the necessity of sourcing cocoa butter, which is often concerned with fluctuations in supply, climate change impacts, and labor-intensive harvesting processes. It also contributes to reducing production costs without compromising quality or sustainability.

As such, according to the present invention, somatic embryos (SEs) are used in food formulations, food products and food materials described herein either comprise these SEs or any valuable extract of such SEs biomass. In some embodiments, the cacao plant cell biomass may be employed as such or directly to manufacture chocolate, chocolate-like products, food products or food materials. In some other embodiments, lipids produced from the SEs biomass, for example, may be extracted from the biomass and utilized to manufacture these products and materials.

The method of the present invention comprises the following steps:
a) culturing an in vitro cacao plant cell biomass in a cell culture medium, optionally with at least one microorganism and/or at least one enzyme, wherein the biomass comprises somatic embryos (SEs) from Theobroma cacao L. explant material;
b) separating the plant cell biomass from the cell culture medium;
c) optionally drying the biomass from step b) at least partially to obtain an at least partially dried biomass;
d) optionally roasting the biomass from step b) or at least partially dried biomass from step c) to obtain a roasted biomass;
e) optionally grinding the biomass from step b), the at least partially dried biomass from step c) or the roasted biomass from step d) to obtain a ground material;
f) optionally conching the ground material from step e) to obtain a conched material; and
g) optionally tempering the ground material from step e) or the conched material from step f).

As used herein, the terms “in vitro” or “in vitro somatic embryogenesis” mean a process constructed in a controlled environment, such as a petri dish, a bioreactor or other artificial conditions.

According to step a), an in vitro cacao plant cell biomass is cultured, optionally with at least one microorganism and/or at least one enzyme, wherein such biomass comprises somatic embryos (SEs) from Theobroma cacao L. explant material.

In some embodiments, step a) consists of culturing a cacao somatic embryo biomass under controlled in vitro conditions to promote cell proliferation and biomass accumulation, optionally introducing, after the growth phase, at least one microorganism and/or at least one enzyme to the cultured biomass.

In some embodiments, step a) consists of incubating the biomass in a liquid medium comprising at least one microorganism and/or at least one enzyme. The liquid medium may comprise one or several microorganisms, one of several enzymes or a combination of both.

During fermentation, the biomass usually reaches a pH ranging between 3.8 and 5.8 which activates enzymes (for example carboxypeptidase and aspartic endoprotease). These enzymes then break down certain proteins to produce free amino acids and oligopeptides. Yeasts and/or bacteria may be added to the biomass, as they contribute to the conversion of sugars to alcohol which helps in the development of lactic acid bacteria. The alcohol is then converted to acetic acid under aerobic conditions and the activity of acetic acid bacteria.

In some embodiments, step a) is a fermentation step and consists of co-culturing the cacao plant cell biomass with at least one microorganism.

Only one microorganism may be used in step a) of the method of the present invention. Alternatively, several microorganisms may be used simultaneously or sequentially in the method of the present invention. For example, step a) may for example consist of co-culturing the biomass with a first microorganism for a determined period of time, then with a second microorganism for another period of time. The microorganism used in the present invention may comprise at least one of yeast, molds, lactic acid bacteria, acetic acid bacteria or other fungi used for enzyme production in food. The microorganism may be specifically selected from the group consisting of yeast, molds, lactic acid bacteria, acetic acid bacteria, other fungi used for enzyme production in food and combinations thereof. The microorganism is preferably selected to produce enzymes effective for the development of flavor, taste and/or aroma; they synthetize and secrete enzymes as part of their metabolic processes.

More precisely, the microorganism may comprise yeast, such as Saccharomyces spp., Candida spp., and Pichia spp., for example, Saccharomyces cerevisiae, Candida milleri, Candida krusei, Pichia kudriavzevii, and Pichia pastoris; molds, such as Aspergillus spp. and Rhizopus spp., for example, Aspergillus niger, Aspergillus oryzae, and Rhizopus stolonifer; bacteria, such as Bacillus spp., for example, Bacillus subtilis and Bacillus pumilus; lactic acid bacteria, such as Leuconostoc spp., Weissella spp., Lactobacillus spp. and Bifidobacterium spp., for example, Leuconostoc mesenteroides, Lactobacillus acidophilus, Lactobacillus plantarum, Lactobacillus fermentum, Lactobacillus brevis, Lactobacillus rhamnosus, and Bifidobacterium bifidum; acetic acid bacteria, such as Acetobacter spp. and Gluconacetobacter spp., for example, Acetobacter aceti, Acetobacter pasteurianus, Gluconobacter oxydans and Gluconacetobacter xylinus; and other fungi used for enzyme production in food, such as Trichoderma spp., Penicillium spp., and Neurospora spp., for example, Trichoderma reesei, Penicillium roqueforti, and Neurospora crassa.

In such embodiments, the microorganism is utilized due to its production of enzymes that contribute to flavor, taste, and/or aroma development. The microorganisms used synthesize and secrete enzymes as part of their metabolic processes.

In some embodiments, step a) comprises the addition of at least one enzyme. The enzymatic treatment with one or several enzymes may be performed in order to generate free amino acids, reducing sugars, and aroma-active compounds.

Only one enzyme may be used in step a) of the method of the present invention. Alternatively, several enzymes may be used simultaneously or sequentially in the method of the present invention. For example, step a) may for example consist of culturing the biomass with a first enzyme for a determined period of time, then with a second enzyme for another period of time. The enzyme used in the present invention may comprise at least one of a protease, a glycosidase, or a lipase. The enzyme used in the present invention may be specifically selected from the group consisting of proteases, glycosidases, lipases and combinations thereof.

The in vitro cacao plant cell biomass of step a) comprises somatic embryos (SEs) from Theobroma cacao L. explant material which may have preferably been produced through at least three successive cycles of direct and/or indirect somatic embryogenesis. Utilizing SEs material which results from at least three successive cycles of direct somatic embryogenesis (DSE), indirect somatic embryogenesis (ISE) or a combination of both may be advantageous, especially if they have been cultured to maximize lipid production, and achieve a lipid content comparable to cacao beans, as described below.

In such embodiments, each cycle in step a) may comprise generating embryos on a first medium and developing such somatic embryos on a second medium, wherein the first medium comprises at least one plant growth regulator (PGR) or hormone and wherein the second medium comprises no PGRs and no hormones, or an amount of PGRs or hormones which is less than 1 wt. %, less than 0.5 wt. %, less than 0.1 wt. % or even less than 0.01 wt. %, based on the total weight of the medium, wherein the first and second media may independently be identical or different for each cycle.

The somatic embryos biomass (or extract) obtained from these embodiments may produce an increased amount of lipids as compared to immature embryos or embryos obtained from the first cycles of somatic embryogenesis, or as compared to any other plant cell material described in the prior art. In some embodiments described below in more detail, the biomass has a lipid content of at least 8 wt. % based on the total weight of the dry biomass and is called “lipid-rich”.

As used herein, the term “lipid-rich” means containing at least 8 wt. % of lipids (or 8% by weight). For example, a lipid-rich biomass or cell extract means respectively a biomass or a cell extract containing at least 8 wt. % of lipids, based on the total weight of the dry biomass or the cell extract. The lipid content of a material is measured on dry materials (dry intact biomass), i.e., by weighing the extracted lipids after the sample has been dried using AOAC 922.06 standard protocol. The lipid content may also, alternatively, be measured according to the method described in Oliva-Cruz M, et al., “Total Fat Content and Fatty Acid Profile of Fine-Aroma Cocoa From Northeastern Peru”. Front Nutr. 2021 Jul. 5; 8:677000. The lipid content may also be measured on wet samples, in case the moisture content of the sample is known (e.g., moisture content of 50 wt. % or 80 wt. %).

As used herein, the term “lipid” includes triglycerides (TG or TAG) and fatty acids (FA), notably saturated, monounsaturated, diunsaturated (diU), polyunsaturated, and trans fatty acids. Lipids may include oleo-palmitostearine, palmitic acid, stearic acid, oleic acid, linoleic acid, linolenic acid, and combinations thereof, for example 1,3-dipalmitoyl-2-oleoyl-glycerol (POP), 1-palmitoyl-2-oleoyl-3-stearoyl-glycerol (POS), and 1,3-distearoyl-2-oleoyl-glycerol (SOS).

The in vitro cacao plant cell biomass of step a) may preferably have a lipid content of at least about 8 wt. % based on the total weight of the dry biomass, for example at least about 10 wt. %, at least about 12 wt. %, at least about 14 wt. % or at least about 15 wt. %.

The at least three cycles of somatic embryos production in combination with the appropriate media environment allow to maximize the production of lipids by the biomass and obtain a cell product having a lipid content comparable to cacao beans, with all the advantages associated.

The variable “n” herein means an integer greater than 3 and the term “nth” refers to the corresponding generation. For example, when n=4, “nth” denotes the fourth generation.

According to an embodiment of step a), somatic embryos (SEs) are produced from Theobroma cacao L. explant material through at least three successive (or consecutive) cycles of direct and/or indirect somatic embryogenesis, in order to produce an in vitro cacao plant cell biomass. The somatic embryos (SEs) may be produced through four, five, six or seven successive (or consecutive) cycles of somatic embryogenesis (SE).

Direct somatic embryogenesis (DSE), indirect somatic embryogenesis (ISE) or a combination of both may be used to prepare the SE biomass of step a). If the preparation of the SEs biomass utilizes several cycles of somatic embryogenesis, each of these cycles may be conducted by direct or indirect somatic embryogenesis, or a combination of both.

Suitable Theobroma cacao L. explant material for use in the present invention may include vegetative or reproductive tissue, or both.

In some embodiments, the tissue comprises zygotic embryos, floral tissue or non-floral tissue, including cacao plant leaves, or a combination thereof. The floral tissue may be selected from the group consisting of petals, sepals, staminodes, and combinations thereof. The non-floral vegetative tissue may be selected from the group consisting of nodes, internodes, young leaves, mature leaves, stems, roots, and combinations thereof.

After being cut or dissected from the T. cacao part, the explant material may be sterilized and/or washed as needed according to the steps described in the literature.

The in vitro lipid-rich cacao plant cell biomass used in step a) of the present invention may be obtained from at least the third generation of the somatic embryos (SEs). For example, somatic secondary embryos (i.e., second generation) are produced from the somatic primary embryos (i.e., first generation). As another example, somatic quaternary embryos (i.e., fourth generation) are produced from the somatic tertiary embryos (i.e., third generation). While genetic stability is a key factor to the methods described in the prior art for plant propagation, the inventors have come to the realization that the possible genetic variation in the somatic embryos used herein does not negatively impact the production and quality of the food products and materials of the present invention. As such, the methods may notably include developing cell lines with a capacity to multiply and grow in size, in order to preferably maximize the production of lipids by the cells.

The cell material (e.g., biomass, cell extract) used in the methods of the invention may be characterized in that they have a lipid content of at least 8 wt. %, based on the total dry weight of the cell material. As described above, the lipid content of a cell material may be determined on dry materials, i.e., by weighing the extracted lipids after the sample has been dried in case of biomass, for example using AOAC 922.06 standard protocol. The lipid content may also be measured on wet samples for example in the case of wet biomass, in case the moisture content of the sample is known (e.g., moisture content of 50 wt. % or 80 wt. %). In some embodiments, the cell material of the present invention has a lipid content of at least 15 wt. %, at least 20 wt. %, at least 25 wt. %, at least 30 wt. %, at least 35 wt. %, at least 40 wt. %, at least 45 wt. %, at least 50 wt. %, at least 55 wt. %, at least 60 wt. %, or at least 65 wt. %, based on the total weight of the cell material.

The plant tissue culture media used to prepare the SE biomass may comprise basal inorganic salts encompassing all essential elements necessary for cellular culture. Typical examples used for somatic embryogenesis include but are not limited to Murashige & Skoog (MS) salts, Driver and Kuniyuki Walnut (DKW) salts and/or Gamborg B-5 salts. Salts can be employed at their full-strength concentration or diluted to varying concentrations to achieve the desired results. In addition, organic molecules such as glycine, myo-inositol, nicotinic acid, pyridoxine, and thiamine, often referred to as “vitamins”, are often added. A fixed carbon source, usually sucrose, is added at concentrations typically between 1.5% and 4% to drive growth. Plant Growth Regulators (PGR) such as auxins, cytokinins, gibberellin, abscisic acid (ABA), jasmonic acid (JA), ethylene, and similar substances, can be added to achieve desired results. The media can be solidified using agar or other polymerizing compounds, or they can be utilized in a liquid state.

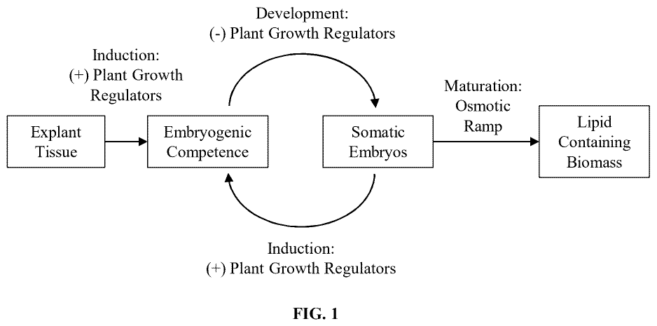

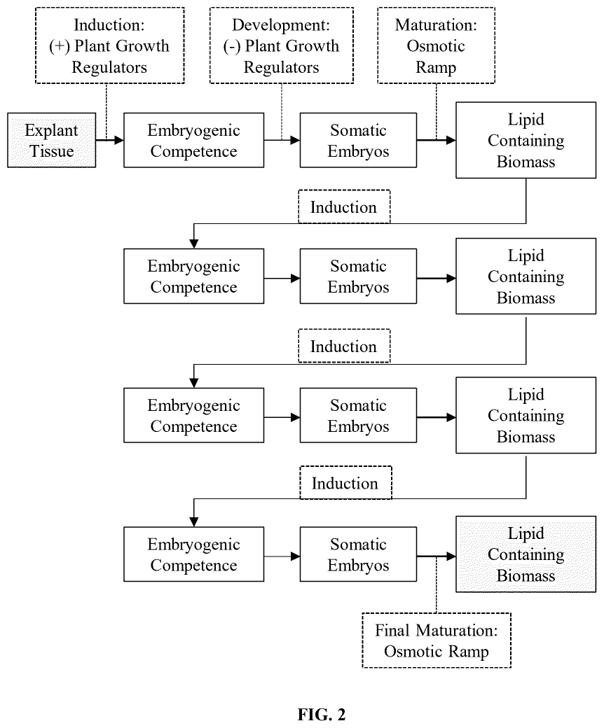

Exemplary methods for preparing the SEs biomass are illustrated in FIG. 1 and FIG. 2. However, it is understood that these methods are not limited to the depicted process.

The culture media used for preparing the SEs biomass may include various media, for example induction media (or inducing media), development media (or developing media) and/or maturation media (maturing media). Some of these media may for example be used sequentially in each cycle of step a).

The compositions of the media for preparing the SEs biomass are not limited herein. They can include salts, vitamins, growth hormones, and an energy source such as glucose, saccharose or sucrose.

In the context of the preparation of the SE biomass concerned with T. cacao somatic embryogenesis, “induction media” are designed to reprogram somatic cells, inducing their transition into a single-cell state or multicellular pro embryonic masses (PEMs) that are capable of following a developmental pattern that mimics the growth of zygotic embryo within a seed. Typically, induction media contain plant growth regulators (PGR) at a sufficient concentration to initiate cellular reprogramming and division of somatic cells within the explant tissue. In some embodiments, induction is accomplished with a single medium composition. In some other embodiments, tissues are incubated sequentially on two or more distinct media compositions to accomplish induction. It should be noted that these subsequent media types are sometimes referred to as “expression media”. In some embodiments, induction media do not support the growth and development of somatic embryos through all the normal morphological stages observed within zygotic embryos (including globular, heart, torpedo, cotyledon).

Specifically, induction media can include one or more plant growth regulators (PGR) or hormones, including, but not limited to, ethephon, kinetin, putrescine, spermidine, hydrogen peroxide, 6-(γ,γ-dimethylallylamino)purine (2iP), and gibberellic acid/gibberellin. The PGR or hormones may be present in concentrations varying from at least 0.01 mg/L to less than 10 mg/L, from example from 0.05 mg/L to 9 mg/L, from 0.10 mg/L to 8 mg/L, from 0.20 mg/L to 7 mg/L, or from 0.50 mg/L to 3.0 mg/L.

In the context of the preparation of the SEs biomass, “development media” may be designed to promote and allow the growth and development of induced cells or PEMs through the normal morphological stages observed within a zygotic embryo. A combination of cell division, cell expansion, and cellular reprogramming leads to the formation of a cotyledon stage somatic embryo that is morphologically and functionally equivalent to a zygotic embryo with an apical meristem, cotyledons, body, and root meristem. Development media differ from induction media in that they contain no PGRs or no hormones, or low PGRs or hormones concentrations. Typically, development media contain no PGRs and no hormones, or an amount of PGRs or hormones which is less than 1 wt. %, less than 0.5 wt. %, less than 0.1 wt. % or even less than 0.01 wt. %, based on the total weight of the medium.

In the context of the preparation of the SEs biomass, “maturation media” may be designed to promote the accumulation of lipids (e.g., triglycerides) and seed storage proteins within the somatic embryo cotyledon tissues. These compounds serve as nutrient and energy storage reserves to sustain early growth of the developing plant immediately after germination. In the present invention, maturation media may contain concentrations of osmoticum, e.g., sugar (such as sucrose, sorbitol, mannitol) or polymeric compounds such as polyethylene glycol (PEG), that increase the osmotic pressure outside the plant cells. This leads to cell dehydration as water leaves the somatic embryo cells through osmosis, cessation of cell division, and biosynthesis of seed storage proteins and lipids. Alternatively, or additionally, the hormone abscisic acid (ABA) which is naturally induced under high osmotic conditions, can be added to the maturation medium (for example in the absence of osmoticum or in combination with high osmotic pressure) to achieve the same result.

In some embodiments of the preparation of the SEs biomass, at least one of the media employed for the preparation of the plant cell biomass used in step a) comprises an amino-acid such as glutamine, proline and arginine, or a derivative thereof; preferably glutamine or a derivative thereof.

In some embodiments, for example illustrated in FIG. 1, each SE cycle may comprise an induction phase during which cells acquire dedifferentiated somatic embryogenic capacity, as well as a development phase. These steps lead to the multiplication of the cells. For example, each cycle may be conducted until the somatic embryos reach maturity. Also illustrated in FIG. 1 is an additional step of maturation, carried out according to this embodiment using an osmotic ramp, as discussed in detail below.

In some embodiments, for example illustrated in FIG. 2, each SE cycle may comprise an induction phase during which cells acquire dedifferentiated somatic embryogenic capacity, as well as a development phase and a maturation phase. More precisely, each cycle comprises a sub-step during which the somatic embryos are cultured under conditions effective to produce a plant cell biomass producing lipids, which may be called maturation phase. The maturation phase for generations 1, 2 and 3, as well as the last maturation phase illustrated on FIG. 2, may be carried out using an osmotic ramp, as discussed below in detail.

According to the methods of the invention, the SE cycles may be independently performed with exposure to light, in the dark or a combination of both. Preferably, the steps are performed in the dark.

According to step a), the somatic embryos are preferably cultured under conditions effective to produce a plant cell biomass which is suitable as a replacement to cocoa butter, e.g., producing lipids. Conditions effective to produce a plant cell biomass suitable as a replacement to cocoa butter, e.g., producing lipids, may preferably comprise an osmoticum.

As used herein, the term “osmoticum” means a substance, combination of substances or solution that influences the movement of water across the plant cell membrane through osmosis, specifically in the context of creating an osmotic gradient. The osmoticum utilized in the present invention may encompass any substance or combination of substances recognized in the field for their capacity to elevate osmotic pressure in the plant cells and/or inhibit the uptake of liquids from the medium, leading to the dehydration/desiccation of the somatic embryos.

In some embodiments, according to step a), the somatic embryos are cultured in the presence of an osmoticum at a concentration effective to produce cell material (e.g., biomass, cell extract) having a lipid content of at least 8 wt. %, based on the total dry weight of the cell material. According to these embodiments, the somatic embryos can be cultured under various osmoticum concentrations.

In some embodiments, the osmoticum comprises at least one of sucrose, maltose, sorbitol, polyethylene glycol (PEG), abscisic acid (ABA), and mixture thereof. For example, the osmoticum may be selected from the group consisting of these substances or their mixtures.

Suspension cell culture may be used in step a) of the method of the present invention for culturing the somatic embryos. Suspension cell culture refers to a method of culturing or growing cells in a liquid medium where the cells are freely suspended and not attached to a substrate (such as the bottom of a culture dish or flask). In contrast to adherent cell culture, where cells attach to a surface, suspension cell culture involves maintaining cells in a state where they float or are suspended in the culture medium. While the term “suspension culture” may be associated with the idea single undifferentiated cell or small aggregates of undifferentiated cells in suspension, this does not apply to the present invention, where the biomass is expected to multiply and grow, for example in the form of aggregates, as an organized tissue and/or as differentiated tissue.

Step a) is preferably carried out in a bioreactor. The bioreactor may for example be chosen among stir tank reactors, stainless steel stirred bioreactors, columns, temporary immersion systems (TIS), air-lift bioreactors, air-culture bioreactors, mist or fog bioreactors, i.e., in bioreactors of 1 to 1000 L, or even more, such as up to 10⁶ L. In some embodiments, step a) is implemented in bioreactors with a volume capacity of at least 1, 10, 25, 50, 75, 100, 500 or 1000 L.

The concentration of the lipids in the material of step a) may be monitored in order to ensure that the cell material meet or remain in the expected specifications, for example that the biomass has a lipid content of at least 8 wt. % based on the total weight of the extract. For example, at an industrial scale, such monitoring may be performed automatically by a control system connected to a computer, in order for example to adjust the composition of the medium according to trajectory setpoints.

According to step b), the plant cell biomass is separated from the cell culture medium. The term “separated” or “separating” should be interpreted in its broadest meaning in the context of the present invention. For example, it may consist in isolating the biomass from the medium. It may also consist in drying the biomass, at least partially.

Such separation step b) is not particularly limited and may include one or several individual sub-steps. In some embodiments, the biomass may be separated from the cell culture medium by filtration, sieving, sedimentation, pressing, decanting, and/or centrifugation. The biomass can be separated from the cell culture medium by draining or extracting at least part of the liquid from the container.

The cell biomass may be characterized by its moisture content. In some embodiments, the moisture content of the biomass remains high, for example of more than 50 wt. %, more than 60 wt. %, or more than 70 wt. %. In some other embodiments, the moisture content of the biomass after step a) is less than 10 wt. %, for example less than 9, 8, 7, 6, 5, 4, 3, 2, 1 or even less than 0.5 wt. %.

In some preferred embodiments, the biomass is dried, at least partially, using techniques such as hot-air or heat drying, vacuum drying, drum drying, rotary drying, freeze-drying or microwave drying. Separating the cells by drying using heat is advantageous, as it accelerates oxidation and increases the sensory and aromatic profiles of the cells. In this case, the temperature of the drying step may vary from 30 to 100° C., for example between 5° and 90° C., for example about 75-85° C.

In some embodiments, the method comprises a further step wherein the biomass is rinsed and washed, for example several times, for example in water, for example in sterile water. This optional step may take place after the separation step b) and/or before the drying step c), if any.

The method of the present invention may also comprise a further step of extraction, or preparation of an in vitro cacao plant cell extract, from the plant cell biomass. It may notably consist in lipid extraction from the SEs biomass. Various extraction techniques may be used for that purpose. For example, horizontal presses may be used for pure lipid extract from the biomass. Alternatively, continuous expeller presses may be used for cocoa butter extraction, followed by solvent extraction from residual cake, and possibly necessitating a refining process. Solvents such as ether, chloroform, and hydrocarbons, in particular hexane and ethanol, can be used in the extraction and refinement of lipid extract from biomass. This optional step may take place after the separation step b) or after the drying step c), if any. In case the method of the invention includes preparing a cacao plant cell extract from the plant cell biomass, this extract may then be mixed with at least one other component. The other steps of the methods, i.e., steps d) to g), remain identical.

Accordingly, in some embodiments, the present invention is directed to a method of producing chocolate, chocolate-like products, food products, or food materials, based on in vitro cacao plant cell biomass, comprising the steps of:
a) culturing an in vitro cacao plant cell biomass in a cell culture medium, optionally with at least one microorganism and/or at least one enzyme, wherein the biomass comprises somatic embryos from Theobroma cacao L. explant material;
b) separating the plant cell biomass from the cell culture medium;
c) optionally drying the biomass from step b) at least partially to obtain an at least partially dried biomass;
c′) producing in vitro cacao plant cell extract from the plant cell biomass of step b) or step c); d) optionally roasting the extract from step c′) to obtain a roasted extract;
e) optionally grinding the extract from step d) to obtain a ground extract;
f) optionally conching the ground extract from step e) to obtain a conched material; and g) optionally tempering the ground material from step e) or the conched material from step f).

In any of the methods of the present invention, according to step c), the biomass from step b) is dried at least partially to obtain an at least partially dried biomass. However, step c) is optional and implemented only if step b) does not use any drying technique. Additionally, step c) may not be needed depending on whether step b) was sufficient to reach the expected moisture content. Both step b) or c) may be implemented, in case step b) does not consist in drying the biomass and depending on the expected moisture content.

Step c) may be carried out using hot-air or heat drying, vacuum drying, drum drying, rotary drying, freeze-drying, microwave drying, or a combination of these methods. The temperature of the drying step may vary from 30 to 100° C., for example between 5° and 90° C., for example about 75-85° C.

According to these embodiments, the cells may be dried until the moisture content is less than about 10 wt. %, for example less than about 9 wt. % or less than about 8 wt. %.

The method of the invention may further comprise a step c″) of exposing or subjecting the biomass (or extract) of step b) or c) to conditions effective to develop taste and/or aroma.

Such conditions effective to develop taste and/or aroma of step c′) may comprise at least one of controlled oxidative conditions, temperature, or humidity conditions effective to develop taste and/or aroma.

More precisely, such conditions effective to develop taste and/or aroma of step c″) may comprise at least one of the following temperature ranges:
A temperature range from about 25 to about 50° C. (i.e., low temperature maturation for slow oxidation and enzyme activity);
A temperature range from about 50 to about 70° C. (i.e., moderate-temperature maturation for accelerated volatile compound formation); and/or
A temperature range from about 70 to about 90° C. (i.e., a high-temperature drying or pre-roasting transition for controlled thermal treatment without full roasting).

The conditions effective to develop taste and/or aroma of step c″) may also comprise subjecting the biomass (or extract) of step b) or c) to a temperature set using hot-air drying, vacuum drying, infrared heating, or fermentation chamber with temperature control preferably for at least about 6 hours.

The conditions effective to develop taste and/or aroma of step c″) may also comprise at least one of the following humidity ranges:
A humidity range of about 10 to about 30% residual humidity (RH) (i.e., a low humidity environment to promote slow drying and/or prevent microbial growth);
A humidity range of about 30 to about 60% RH (i.e., a moderate humidity environment to allows gradual precursor formation while maintaining moisture for enzymatic reactions); and/or
A humidity range of about 60 to about 85% RH (i.e. a high humidity environment to support enzymatic activity, fermentation-like conditions, and/or controlled oxidation).

The conditions effective to develop taste and/or aroma of step c″) may also comprise subjecting the biomass (or extract) of step b) or c) to a humidity set using a humidity-controlled chamber, humidified air stream, and/or wet aging techniques.

The conditions effective to develop taste and/or aroma of step c″) may also comprise subjecting the biomass (or extract) of step b) or c) to a catalyst, preferably a polyphenol oxidase (PPO) to enhance oxidation, a catalase to regulate oxidative stress and/or an enzyme deactivation step if oxidation needs to be halted.

The conditions effective to develop taste and/or aroma of step c″) may be implemented for a duration of between about 6 to about 96 hours, between about 12 to about 96 hours or between about 12 and about 72 hours.

The method of the present invention may further comprise the addition of at least one of a reducing sugar, an amino acid or a polyphenolic compound, prior to step d). The chemical precursor enhancement is performed by incorporating reducing sugars, or polyphenolic compounds to drive Maillard reaction pathways, prior to the roasting of step d).

The method of the present invention may also further comprise the addition of at least one volatile compound, prior to step d). The term “volatile compound” refers to a flavor or aroma compound or a chocolate-associated compound. Such compound preferably comprises at least one of linalool, furaneol or a methylpyrazine.

According to step d), the biomass from step b) or at least partially dried biomass from step c) of the extract from step c′) is roasted, to obtain a roasted biomass (or extract). This step is optional.

Roasting is typically conducted at high temperatures. Several types of reactions generally occur during roasting (i.e., Maillard reaction, Amadori rearrangement, and Strecker-type reactions), contributing to the complex mixture of compounds (including pyrazines, furans, pyrroles, aldehydes, ketones, thiophenes, phenols), which creates a unique flavor and aroma profile.

Step d) may be carried out using drum roasting, air roasting, tangential roasting, packed bed roasting, continuous roasting, batch roasting, infrared roasting, continuous belt roasting, centrifugal roasting, or a combination of these methods.

Also, step d) may be carried out in an oven at temperatures ranging between about 100 and about 190° C. for about 1 minute to about 30 minutes.

According to step e), the biomass from step b), at least partially dried biomass from step c) or the roasted biomass (or extract) from step d) is ground to obtain a ground material. This step is optional.

Any method suitable for achieving the desired particle size and texture may be employed, including both traditional and modern grinding techniques such as stone grinding, ball milling, and roller refining.

Step e) may be carried out using stone grinding, ball milling, roller refining, or a combination of these methods.

According to step f), the ground material from step e) is conched, to obtain a conched material. This step is optional. Step f) may not be needed depending on step e), especially if step e) is carried out using stone grinding, ball milling, roller refining, or a combination of these methods.

Any conching methods suitable for homogenizing the chocolate material and refining the flavor may be employed, including long-duration conching, short-duration conching, high-shear conching, low-shear conching, or a combination of these methods.

Step f) may be carried out using long-duration conching, short-duration conching, high-shear conching, low-shear conching, or a combination of these methods.

According to step g), the ground material from step e) or the conched material from step f) is tempered. This step is optional.

Tempering is a process of controlled heating and cooling that stabilizes the chocolate or chocolate-like products and gives it a smooth, shiny appearance. Tempering is described in the literature. It is preferably performed by heating the product to a specific temperature and then cooling it down while stirring constantly.

Any tempering method suitable for crystallizing the chocolate material to improve texture and stability may be used, including methods such as tabling, seeding, or machine tempering.

Step g) may be carried out using tabling, seeding, machine tempering, or a combination of these methods.

Step g) may be carried out using cocoa silk. Cocoa silk is a solid or soft paste made of 100% cocoa butter that is in the proper tempered form (Beta Form V). According to this embodiment, cocoa silk is added to the melted ground material from step e) or the conched material from step f) in order to seed or temper such material by providing a framework on which the remaining material crystals form.

The method of the present invention may further comprise a step d′) of adding at least one other component to the biomass of step b), the at least partially dried biomass of step c) or to the roasted biomass (or extract) of step d), wherein the other component preferably comprises at least one of carbohydrates, sweeteners, vegetable fats, structuring agents, milk solids, alternative milk solids, emulsifiers, aroma, prebiotic fibers, or cocoa pulp.

The other component may be selected from the group consisting of cocoa butter, alternative plant-based fats (e.g., shea butter, mango butter, sunflower lecithin), structuring agents (e.g., mono- and diglycerides), sweeteners & functional sugars (to improve taste & solubility), natural sweeteners (e.g., honey, maple syrup, agave syrup), alternative sugars (e.g., allulose, erythritol, monk fruit extract), prebiotic fibers and lecithin.

The other (or added) component(s) may constitute between about 0.1 wt. % to about 80 wt. % of the product or material, for example between about 5 and about 50 wt. % or between about 10 and about 40 wt. %.

In some preferred embodiments, the method of the invention comprises fermentation (i.e., co-culture) of the SEs biomass with at least one microorganism, preferably yeast, using a fermentation media comprising sucrose, sugar and glucose. In these embodiments, the method of the invention also comprises at least one of drying, roasting, grinding and tempering, and preferably all these steps. In this method, the SEs biomass preferably has a lipid content of at least 8 wt. % and/or has been produced through at least three successive cycles of direct and/or indirect somatic embryogenesis, as described herein.

Although usable on a smaller scale, the methods of the invention are preferably implemented on an industrial scale for commercial production.

The present invention is also directed to chocolate, chocolate-like products, food products or food materials. The products and materials may be obtained from the methods described herein.

In some embodiments, these products or materials are cocoa powder, cocoa butter or chocolate.

Unfortunately, the soil heavy metal accumulation leads to farm products presenting undesirable heavy metal contents. In contrast, the chocolate or chocolate-like products of the present invention may be advantageously characterized by their low content in heavy metals, including Cadmium (Cd) and Lead (Pb). Notably, they may be characterized by their content in Cd and/or Pb in the biomass being less than 10 ppb, less than 5 ppb, less than 1 ppb or even less than 0.5 ppb, based on the total weight of the dry biomass. Cd and Pb contents may be measured by Inductively Coupled Plasma Mass Spectrometry (ICP-MS) and by Inductively Coupled Plasma Optical Emission Spectrometry (ICP-OES). In the ICP-MS method, the prepared sample is aerosolized and introduced into a high-temperature plasma, where the atoms are ionized. The mass spectrometer then separates and quantifies the ions based on their mass-to-charge ratio. In the ICP-OES method, inductively coupled plasma is used to atomize the sample. The emitted light at characteristic wavelengths is then measured to determine the concentration of cadmium. Reference may be made to the article of Martin Rose, et al., “A Review of Analytical Methods for Lead, Cadmium, Mercury, Arsenic and Tin Determination Used in Proficiency Testing”, J. Anal. At. Spectrom., 2001,16, 1101-1106.

Similarly as for the biomass, the plant cell biomass or extract used to prepare the products and materials of the invention may be advantageously characterized by its low content in heavy metals, including Cadmium (Cd) and Lead (Pb). For example, these products or materials may be characterized in that their content in Cd and/or Pb is less than 10 ppb, less than 5 ppb, less than 1 ppb or even less than 0.5 ppb, based on the total weight of the product or material.

The chocolate, chocolate-like products, food products, or food materials, may be characterized in that they comprise somatic embryos (SEs) or an SEs extract from Theobroma cacao L. explant material, wherein the SEs have preferably been produced through at least three successive cycles of direct and/or indirect somatic embryogenesis. In these embodiments, the SEs may have been produced through at least four, five or six successive cycles of direct and/or indirect somatic embryogenesis.

The SEs may be characterized in that they have a lipid content of at least about 8 wt. % based on the total weight of the dry SEs biomass, for example at least about 10 wt. %, at least about 12 wt. %, at least about 14 wt. % or at least about 15 wt. %.

In these embodiments, the SEs biomass (or extract) produces an increased amount of lipids as compared to immature embryos or embryos obtained from the first cycles of somatic embryogenesis, or as compared to any other plant cell material described in the prior art (experimental data below).

The products or materials of the present invention may comprise at least one additional component, wherein the component may preferably comprise or be selected among at least one of carbohydrates, sweeteners, vegetable fats, structuring agents, milk solids, alternative milk solids, emulsifiers, aroma, prebiotic fibers, or cocoa pulp. Combinations of these components are also possible.

The added component(s) may constitute between about 0.1 wt. % to about 80 wt. % of the finished product, for example between about 5 and about 50 wt. % or between about 10 and about 40 wt. %.

The product of the present invention may be a dairy, for example, chocolate milk, chocolate-flavored yogurt, chocolate cheese spreads, chocolate pudding, and chocolate cream; a frozen dessert, for example, chocolate ice cream, chocolate gelato, chocolate sorbet, chocolate frozen yogurt, chocolate mousse, and chocolate-covered ice cream bars; a baked good, for example, chocolate cake, chocolate chip cookies, chocolate muffins, brownies, chocolate croissants, chocolate-filled pastries, chocolate tarts, and chocolate eclairs; or a beverage, for example, hot chocolate, mocha coffee, chocolate milkshakes, chocolate protein shakes, chocolate smoothies, and chocolate-infused liqueurs.

The present invention is also directed to the use of somatic embryos (SE) from Theobroma cacao L. explant material to produce chocolate, chocolate-like or food products, or food materials, wherein the SE have been preferably produced through at least three successive cycles of direct and/or indirect somatic embryogenesis.

In such embodiments, the SEs may have been produced through at least four, five or six successive cycles of direct and/or indirect somatic embryogenesis.

The SEs may preferably have a lipid content of at least about 8 wt. % based on the total weight of the dry SEs biomass, for example at least about 10 wt. %, at least about 12 wt. %, at least about 14 wt. % or at least about 15 wt. %.

All the features described herein in connection with the methods and products of the present invention also apply to such uses for producing the products and materials.

### Aspects of the Invention

The invention may be according to the following aspects.
Aspect 1. A method of producing chocolate, chocolate-like or food products, or food materials, based on in vitro cacao plant cell biomass, comprising the steps of:
   a) culturing an in vitro cacao plant cell biomass in a cell culture medium, optionally with at least one microorganism and/or at least one enzyme, wherein the biomass comprises somatic embryos from Theobroma cacao L. explant material;
   b) separating the plant cell biomass from the cell culture medium;
   c) optionally drying the biomass from step b) at least partially to obtain an at least partially dried biomass;
   d) optionally roasting the biomass from step b) or at least partially dried biomass from step c) to obtain a roasted biomass;
   e) optionally grinding the biomass from step b), the at least partially dried biomass from step c) or the roasted biomass from step d) to obtain a ground material;
   f) optionally conching the ground material from step e) to obtain a conched material; and
   g) optionally tempering the ground material from step e) or the conched material from step f).
Aspect 2. The method of aspect 1, wherein step a) consists of:
   culturing a cacao somatic embryo biomass under controlled in vitro conditions to promote cell proliferation and biomass accumulation, optionally introducing, after the growth phase, at least one microorganism and/or at least one enzyme to the cultured biomass; or
   co-culturing the cacao plant cell biomass with at least one microorganism, wherein the microorganism preferably comprises at least one of yeast, molds, bacteria, lactic acid bacteria, acetic acid bacteria or other fungi used for enzyme production in food;
   wherein the microorganism preferably comprises at least one of the followings:
   yeast, such as Saccharomyces spp., Candida spp., and Pichia spp., for example, Saccharomyces cerevisiae, Candida milleri, Candida krusei, Pichia kudriavzevii, and Pichia pastoris; molds, such as Aspergillus spp. and Rhizopus spp., for example, Aspergillus niger, Aspergillus oryzae, and Rhizopus stolonifer;
   bacteria, such as Bacillus spp., for example, Bacillus subtilis and Bacillus pumilus;
   lactic acid bacteria, such as Leuconostoc spp., Weissella spp., Lactobacillus spp. and Bifidobacterium spp., for example, Leuconostoc mesenteroides, Lactobacillus acidophilus, Lactobacillus plantarum, Lactobacillus fermentum, Lactobacillus brevis, Lactobacillus rhamnosus, and Bifidobacterium bifidum;
   acetic acid bacteria, such as Acetobacter spp. and Gluconacetobacter spp., for example, Acetobacter aceti, Acetobacter pasteurianus, Gluconobacter oxydans and Gluconacetobacter xylinus; and
   other fungi used for enzyme production in food, such as Trichoderma spp., Penicillium spp., and Neurospora spp., for example, Trichoderma reesei, Penicillium roqueforti, and Neurospora crassa.
Aspect 3. The method of aspect 1 or 2, wherein step a) consists of incubating the biomass in a liquid medium comprising at least one microorganism and/or at least one enzyme.
Aspect 4. The method of any one of aspects 1-3, wherein step a) comprises the addition of at least one enzyme, wherein the enzyme preferably comprises at least one of a protease, a glycosidase, or a lipase.
Aspect 5. The method of any one of aspects 1-4, further comprising the addition of at least one of a reducing sugar, an amino acid or a polyphenolic compound, prior to step d).
Aspect 6. The method of any one of aspects 1-5, further comprising the addition of at least one volatile compound, prior to step d), wherein the compound preferably comprises at least one of linalool, furaneol or a methylpyrazine.
Aspect 7. The method of any one of aspects 1-6, wherein the microorganism produces enzymes effective for the development of flavor, taste and/or aroma.
Aspect 8. The method of any one of aspects 1-7, wherein step c) is carried out using hot-air or heat drying, vacuum drying, drum drying, rotary drying, freeze-drying, microwave drying, or a combination of these methods.
Aspect 9. The method of any one of aspects 1-8, further comprising a step c′) of exposing or subjecting the biomass of step b) or c) to conditions effective to develop taste and/or aroma.
Aspect 10. The method of aspect 9, wherein the conditions effective to develop taste and/or aroma of step c′) comprise at least one of controlled oxidative conditions, temperature, or humidity conditions effective to develop taste and/or aroma.
Aspect 11. The method of any one of aspects 1-10, wherein the conditions effective to develop taste and/or aroma of step c′) comprise at least one of the following temperature ranges:
   A temperature range from about 25 to about 50° C. (i.e., low temperature maturation for slow oxidation and enzyme activity);
   A temperature range from about 50 to about 70° C. (i.e., moderate-temperature maturation for accelerated volatile compound formation); and/or
   A temperature range from about 70 to about 90° C. (i.e., a high-temperature drying or pre-roasting transition for controlled thermal treatment without full roasting).
Aspect 12. The method of any one of aspects 1-11, wherein the conditions effective to develop taste and/or aroma of step c′) comprise subjecting the biomass of step b) or c) to a temperature set using hot-air drying, vacuum drying, infrared heating, or fermentation chamber with temperature control preferably for at least about 6 hours.
Aspect 13. The method of any one of aspects 1-12, wherein the conditions effective to develop taste and/or aroma of step c′) comprise at least one of the following humidity ranges:
   A humidity range of about 10 to about 30% residual humidity (RH) (i.e., a low humidity environment to promote slow drying and/or prevent microbial growth);
   A humidity range of about 30 to about 60% RH (i.e., a moderate humidity environment to allows gradual precursor formation while maintaining moisture for enzymatic reactions); and/or
   A humidity range of about 60 to about 85% RH (i.e. a high humidity environment to support enzymatic activity, fermentation-like conditions, and/or controlled oxidation).
Aspect 14. The method of any one of aspects 1-13, wherein the conditions effective to develop taste and/or aroma of step c′) comprise subjecting the biomass of step b) or c) to a humidity set using a humidity-controlled chamber, humidified air stream, and/or wet aging techniques.
Aspect 15. The method of any one of aspects 1-14, wherein the conditions effective to develop taste and/or aroma of step c′) comprise subjecting the biomass of step b) or c) to a catalyst, preferably a polyphenol oxidase (PPO) to enhance oxidation, a catalase to regulate oxidative stress and/or an enzyme deactivation step if oxidation needs to be halted.
Aspect 16. The method of any one of aspects 1-15, wherein the conditions effective to develop taste and/or aroma of step c′) are implemented for a duration of between about 6 to about 96 hours, between about 12 to about 96 hours or between about 12 and about 72 hours.
Aspect 17. The method of any one of aspects 1-16, wherein step d) is carried out using drum roasting, air roasting, tangential roasting, packed bed roasting, continuous roasting, batch roasting, infrared roasting, continuous belt roasting, centrifugal roasting, or a combination of these methods.
Aspect 18. The method of any one of aspects 1-17, wherein step d) is carried out in an oven at temperatures ranging between about 100 and about 190° C. for about 1 minute to about 30 minutes.
Aspect 19. The method of any one of aspects 1-18, wherein step e) is carried out using stone grinding, ball milling, roller refining, or a combination of these methods.
Aspect 20. The method of any one of aspects 1-19, wherein step f) is carried out using long-duration conching, short-duration conching, high-shear conching, low-shear conching, or a combination of these methods.
Aspect 21. The method of any one of aspects 1-20, wherein step g) is carried out using tabling, seeding, machine tempering, or a combination of these methods.
Aspect 22. The method of any one of aspects 1-21, further comprising a step d′) of adding at least one other component to the roasted biomass of step d), wherein the other component preferably comprises at least one of carbohydrates, sweeteners, vegetable fats, structuring agents, milk solids, alternative milk solids, emulsifiers, aroma, prebiotic fibers, or cocoa pulp.
Aspect 23. The method of aspect 22, wherein the added component(s) constitute between about 0.1 wt. % to about 80 wt. % of the finished product, for example between about 5 and about 50 wt. % or between about 10 and about 40 wt. %.
Aspect 24. The method of any one of aspects 1-23, wherein step a) is at least partially carried out in a bioreactor, preferably in a stir tank reactor, a temporary immersion system (TIS), an air-lift bioreactor, an air-culture bioreactor, or a mist or fog bioreactor.
Aspect 25. The method of any one of aspects 1-24, wherein the in vitro cacao plant cell biomass of step a) comprises somatic embryos from Theobroma cacao L. explant material produced through at least three successive cycles of direct and/or indirect somatic embryogenesis.
Aspect 26. The method of aspect 25, wherein each cycle in step a) comprises generating embryos on a first medium and developing such somatic embryos on a second medium, wherein the first medium comprises at least one plant growth regulator (PGR) or hormone and wherein the second medium comprises no PGRs and no hormones, or an amount of PGRs or hormones which is less than 1 wt. %, less than 0.5 wt. %, less than 0.1 wt. % or even less than 0.01 wt. %, based on the total weight of the medium, wherein the first and second media may independently be identical or different for each cycle.
Aspect 27. The method of any one of aspects 1-26, wherein the in vitro cacao plant cell biomass of step a) has a lipid content of at least about 8 wt. % based on the total weight of the dry biomass, for example at least about 10 wt. %, at least about 12 wt. %, at least about 14 wt. % or at least about 15 wt. %.
Aspect 28. Chocolate, chocolate-like products, food products or food materials obtained from the method of any one of aspects 1-27.
Aspect 29. The products or materials of aspect 28, being cocoa powder, cocoa butter or chocolate.
Aspect 30. The products or materials of aspect 28 or 29, characterized in that their content in Cd and/or Pb is less than 10 ppb, less than 5 ppb, less than 1 ppb or even less than 0.5 ppb, based on the total weight of the product.
Aspect 31. Chocolate, chocolate-like products, food products, or food materials, comprising somatic embryos (SEs) from Theobroma cacao L. explant material, wherein the SEs have preferably been produced through at least three successive cycles of direct and/or indirect somatic embryogenesis.
Aspect 32. The products or materials of aspect 31, wherein the SEs have been produced through at least four, five or six successive cycles of direct and/or indirect somatic embryogenesis.
Aspect 33. The products or materials of any aspects 31-32, wherein the SEs have a lipid content of at least about 8 wt. % based on the total weight of the dry SEs biomass, for example at least about 10 wt. %, at least about 12 wt. %, at least about 14 wt. % or at least about 15 wt. %.
Aspect 34. The products or materials of any aspects 28-33, comprising at least one component, wherein the component comprises at least one of carbohydrates, sweeteners, vegetable fats, structuring agents, milk solids, alternative milk solids, emulsifiers, aroma, prebiotic fibers, or cocoa pulp.
Aspect 35. The products or materials of aspect 34, wherein the added component(s) constitute between about 0.1 wt. % to about 80 wt. % of the finished product, for example between about 5 and about 50 wt. % or between about 10 and about 40 wt. %.
Aspect 36. The products or materials of any aspects 28-35, wherein the product is a dairy, for example, chocolate milk, chocolate-flavored yogurt, chocolate cheese spreads, chocolate pudding, and chocolate cream; a frozen dessert, for example, chocolate ice cream, chocolate gelato, chocolate sorbet, chocolate frozen yogurt, chocolate mousse, and chocolate-covered ice cream bars; a baked good, for example, chocolate cake, chocolate chip cookies, chocolate muffins, brownies, chocolate croissants, chocolate-filled pastries, chocolate tarts, and chocolate eclairs; or a beverage, for example, hot chocolate, mocha coffee, chocolate milkshakes, chocolate protein shakes, chocolate smoothies, and chocolate-infused liqueurs.
Aspect 37. Use of somatic embryos (SE) from Theobroma cacao L. explant material to produce chocolate, chocolate-like or food products, or food materials, wherein the SE have been preferably produced through at least three successive cycles of direct and/or indirect somatic embryogenesis.
Aspect 38. Use of aspect 37, wherein the SEs have been produced through at least four, five or six successive cycles of direct and/or indirect somatic embryogenesis.
Aspect 39. Use of any aspects 37-38, wherein the SEs have a lipid content of at least about 8 wt. % based on the total weight of the dry SEs biomass, for example at least about 10 wt. %, at least about 12 wt. %, at least about 14 wt. % or at least about 15 wt. %.
Aspect 40. Use of any aspects 37-39, comprising at least one component, wherein the component comprises at least one of carbohydrates, sweeteners, vegetable fats, structuring agents, milk solids, alternative milk solids, emulsifiers, aroma, prebiotic fibers, or cocoa pulp.
Aspect 41. Use of aspect 40, wherein the added component(s) constitute between about 0.1 wt. % to about 80 wt. % of the finished product, for example between about 5 and about 50 wt. % or between about 10 and about 40 wt. %.
Aspect 42. Use of any aspects 37-41, wherein the product is a dairy, for example, chocolate milk, chocolate-flavored yogurt, chocolate cheese spreads, chocolate pudding, and chocolate cream; a frozen dessert, for example, chocolate ice cream, chocolate gelato, chocolate sorbet, chocolate frozen yogurt, chocolate mousse, and chocolate-covered ice cream bars; a baked good, for example, chocolate cake, chocolate chip cookies, chocolate muffins, brownies, chocolate croissants, chocolate-filled pastries, chocolate tarts, and chocolate eclairs; or a beverage, for example, hot chocolate, mocha coffee, chocolate milkshakes, chocolate protein shakes, chocolate smoothies, and chocolate-infused liqueurs.

### EXAMPLES

The following examples are given for the purpose of illustrating various embodiments of the disclosure and are not meant to limit the present disclosure in any fashion. The present examples, along with the methods described herein are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### Media Compositions

Chemicals from Sigma Aldrich were used for all media preparation, including a powdered form of the DKW medium.

TABLE 1
Media A-H

| Media | A | B | C | D | E | F | G | H |
|---|---|---|---|---|---|---|---|---|
| DKW Salts | 1x | | 1x | 1x | 1x | 1x | 1x | 1x |
| DKW Vitamins | 1x | | 1x | 1x | 1x | 1x | 1x | 1x |
| McCown WPM Salts | | 1x | | | | | | |
| Gamborg B5 Vitamins | | 1x | | | | | | |
| myo-inositol (mg/L) | 100 | | | | | | | |
| L-glutamine (mg/L) | 250 | | | 1,000 | 1,000 | 1,000 | 1,000 | 1,000 |
| TDZ (mg/L) | 0.005 | | | | | | | |
| BAP (mg/L) | | 0.05 | | | | | | |
| 2,4-D (mg/L) | 2 | 2.0 | | | | | | |
| Glucose (g/L) | 20 | 20 | | | | | | |
| Sucrose (g/L) | | | 40 | 30 | 90 | 150 | 210 | 270 |

TABLE 2
Media E2-H2

| Media | E2 | F2 | G2 | H2 |
|---|---|---|---|---|
| DKW Salts | 1x | 1x | 1x | 1x |
| DKW Vitamins | 1x | 1x | 1x | 1x |
| L-glutamine (mg/L) | 1,000 | 1,000 | 1,000 | 1,000 |
| Mannitol (g/L) | 32 | 64 | 96 | 128 |
| Sucrose (g/L) | 30 | 30 | 30 | 30 |

TABLE 3
Media J1-J5

| Media | J1 | J2 | J3 | J4 | J5 |
|---|---|---|---|---|---|
| MS Salts | 1x | 1x | 1x | 1x | 1x |
| MS Vitamins | 1x | 1x | 1x | 1x | 1x |
| Casein hydrolysate (g/L) | 1.0 | 1.0 | 1.0 | 1.0 | 1.0 |
| Sucrose (g/L) | 30 | 90 | 150 | 210 | 270 |

TABLE 4
Media X, Y and Z

| | Media X | Media Y | Media Z |
|---|---|---|---|
| MS Salts | 1x | 1x | 1x |
| MS Vitamins | 1x | 1x | 1x |
| L-glutamine (mg/L) | 250 | 250 | 250 |
| Myo-inositol (mg/L) | 100 | 100 | 100 |
| Sucrose (g/L) | 30 | 30 | 30 |
| Zeatin (mg/L) | 0.2 | 0.6 | 0.4 |
| IAA (mg/L) | 0.2 | 0.6 | 2.0 |
| gelzan (g/L) | 2.0 | 2.0 | — |

### Example 1—Induction of Primary Somatic Embryos (PSEs) from Flower and Immature Zygotic Tissues

Whole cacao pods and immature flower buds were first surface disinfected before being processed aseptically. Exteriors of pods were scrubbed in commercial detergent solution then rinsed in water. All plant material was then rinsed in alcohol, washed in a sodium hypochlorite plus Triton™ X100 solution, and rinsed several times in sterile water.

For extraction of immature embryos, surface-sterilized pods were cut into quarters and beans were exposed. Pulp and seed coats were aseptically removed from beans and 0.5 to 2.0 cm immature embryos were extracted as induction material. Whole staminodes and petals were separated from bases of flower buds. Zygotic tissues and petals were cut into 1-3 mm pieces. Staminodes were cultured intact.

All explants were first cultured onto petri dishes of Media A for two weeks. Explants were then transferred onto a sequence of media replaced every two weeks: Media B, Media C, Media D. Thereafter, explants were maintained on Media D, subculturing every two weeks for up to an additional four months. PSEs began appearing asynchronously from 8-10 weeks in culture. As they developed, PSEs were left untouched on source explants until they reached torpedo stage and easily separated from source tissue. All cultures were incubated in darkness at 26° C.

### Example 2—Induction of Secondary, Tertiary and Quaternary Somatic Embryos (SSEs, TSEs, QSEs)

For induction of SSEs, PSEs maintained on Media D were selected based on maturity and appearance. PSEs between 4-12 mm long and newly developing pink pigment were chosen for culture. Cotyledons were separated from the embryo body and cut into 1-3 mm pieces. Cut explants were cultured following the methods of PSE induction as described in Example 1 except omitting Media A. After 6-8 weeks, cotyledons from developing SSEs were cultured as before to generate a tertiary cycle of SEs (TSEs). With the same methods, the fourth, quaternary cycle of SEs (QSEs) was then generated from cotyledonary explants from TSEs. Up to 364 embryos could be derived per single induced embryo.

### Example 3—Maturation of SEs for Cocoa Lipids

Embryos from QSEs were used for liquid culture maturation. More precisely, white, unpigmented embryos ranging between heart to cotyledonary-stage were used.

SEs were cultured over 42 days through an osmoticum ramp composed of one of three sets of media applied in sequence: Medias D through H, Medias E2 through H2, or Medias J1 through J5. Cultures were maintained for 10 days on Media D or J1, for 2 days each on Medias E, E2 or J2 through G, G2 or J4, followed by 26 days on Media H, H2 or J5.

Media D through Media H and Media E2 through Media H2 were composed of Driver and Kuniyuki Walnut (DKW) salts and vitamins supplemented with 1.0 g/L L-glutamine and either sucrose (Media D through Media H) or an equimolar mixture of sucrose and mannitol (Media E2 through Media H2). Media E through H were identical to Media D, but contain 9%, 15%, 21% or 27% sucrose. Media J-1 through Media J-5 were composed of Murashige & Skoog (MS) salts and vitamins supplemented with 1 g/L casein hydrolysate and 3% to 27% sucrose.

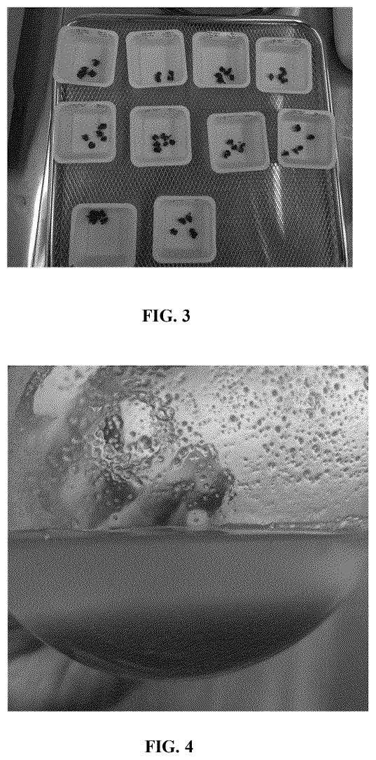

Five embryos per flask were cultured in 8 mL liquid media in 250 mL Erlenmeyer flasks and incubated at 26° C. Flasks were kept under 60 RPM agitation in darkness. After the first 16 days of culture, media was replaced weekly. After 42 days in culture, embryo fresh and dry weights were collected. See FIG. 3. Cultures produced a mean dry weight (DW) of 85.6 mg per embryo (SD=31.8) and mean dry mass of 25.9%.

### Example 4—Comparative—Establishment of Callus and Liquid Cell Suspension (No Somatic Embryos)

Whole cacao pods were disinfected then processed aseptically. Pods were scrubbed in detergent solution then rinsed in water, soaked in alcohol, washed in a bleach-detergent solution, then rinsed again in sterile water. Pods were then cut into quarters to expose beans for extraction and culture.

For the establishment of callus, mature beans were chopped into 2-3 mm pieces and cultured onto petri dishes containing 50 mL of Media X. Cultures were incubated in darkness at 26° C. transferring to fresh media every two weeks. After 3 months, callus explants were then maintained on Media Y for another 3 months under the same conditions.

For establishment of cell suspensions, appropriate callus was cultured into 25 to 100 mL of Media Z in Erlenmeyer flasks. Suspensions were inoculated to approximately 10% cell density and incubated at 26° C. in darkness on an incubator-shaker set to 100 RPM agitation. During each subculture, culture cell density was re-adjusted to 10%. Subcultures were carried out regularly on a weekly basis. Suspension cultures were maintained under these conditions for more than 6 months. See. FIG. 4.

### Example 5—Total Fat Analysis of T. cacao Somatic Embryo Culture Biomass

Total fat content of SE biomass is presented with data from suspension culture cell biomass for comparison.

For analysis, 15 grams of dried cultured biomass from example 3 (inventive) was analyzed for fat content analysis using AOAC 922.06 standard protocol.

Also, 400 mL of cell suspension (comparative) was washed with distilled water through a Buchner funnel. Cell mass was collected and analyzed for fat content analysis using AOAC 922.06 standard protocol.

### Results

TABLE 5
Lipid Content

| | SE Culture Biomass | Liquid Cell Suspension |
|---|---|---|
| | (inventive) | Biomass (comparative) |
| Lipid Content (%) | 12.4 | 1.6 |

Fat and fatty acid analysis of T. cacao SEs indicates that the current cell line is capable of producing greater than 10% lipids on a dry weight basis, while cell suspension biomass produces only 1.6% lipids.

### Example 6—Multiplication Rate

To estimate multiplication rate (MR), embryos were counted by hand and MR calculated using the modified standard growth equation from Firoozabady, et al., “Cost-effective in vitro propagation methods for pineapple.” Plant Cell Reports 21 (2003): 844-850.

Embryo weight data was calculated from control treatment results across several experiments to estimate average fresh (FW) and dry weight (DW). Summary tables of MR and weight are included below.

TABLE 6
Number of new tertiary somatic embryos produced per single secondary
somatic embryo (n = 53 secondary somatic embryos cultured)

| | New SE generated per source SE |
|---|---|
| AVG | 127.28 |
| SD | 66.75 |

TABLE 7
Fresh and dry weight per single somatic embryo after osmotic
ramp maturation (n = 181 somatic embryos cultured)

| | FW/SE (mg) | DW/SE (mg) |
|---|---|---|
| AVG | 261 | 86 |
| SD | 135 | 30 |

FW = fresh weight - embryo mass after osmotic ramp maturation
DW = dry weight - embryo mass after overnight dehydration

### Example 7—Chocolate Manufacturing

### Co-Culture—Fermentation

Fermentation was conducted in a FastRack one-gallon (3.79 liters) wide-mouth jar equipped with a drilled lid and twin bubble airlock. The fermentation media was prepared by combining distilled water, sucrose, dark brown sugar, glucose, and WLP546 Maran6n Canyon Wild Cacao Yeast from White Labs. The mixture was warmed below 32° C. to dissolve sugars and cooled below 29° C. before yeast inoculation. Prior to use, all equipment was sterilized.

After media preparation, 119 g of in vitro cacao plant cell biomass was drained and transferred into the fermentation vessel. Fermentation proceeded for five days, with stirring every 5-6 hours to enhance intracellular compound breakdown into flavor precursor molecules such as organic acids and reducing sugars.

### Fermentation Parameters

TABLE 8
Temperature Profile During Fermentation

| Time (Hours) | Temperature (° F.) |
|---|---|
| 0 | 75 |
| 24 | 85 |
| 48 | 86 |
| 72 | 85 |
| 86 | 86 |
| Harvest | 78 |

TABLE 9
Sugar Content Analysis

| Time Point | Sugar Content (%) |
|---|---|
| Initial (0 hours) | 44.5 |
| End of Fermentation (Harvest) | 26.1 |

TABLE 10
Fermentation Media pH Analysis

| Time Point | pH Value |
|---|---|
| Initial Stage | 5.75 |
| Ending Stage | 4.54 |

The observed decrease in pH from 5.75 to 4.54 indicates active microbial metabolism and organic acid production during fermentation.

### Separation

After fermentation, the biomass was separated from the media using a chinois lined with cheesecloth.

### Drying

Cells were dehydrated in an Excalibur 9-Tray Food Dehydrator for 2 days at 68° C.

### Roasting

The dried cells were roasted using a Barwell Coffee Roaster:
20 min at 180° C. (initial and final blast stages);
20 min at 160° C.; and
30 min at 140° C.

### Grinding

The roasted biomass was ground in a Premier Chocolate Refiner with 145 g melted cocoa butter and 70 g sucrose. Particle size reduction was monitored using a Spectra Micron Meter, with grinding continued until the chocolate liquor reached 15 microns.

TABLE 11
Evolution of the particle size over time

| Time (Hours) | Particle Size (Microns) |
|---|---|
| 12 | 90 |
| 20 | 25 |
| 24 | 15 |

### Tempering

The chocolate liquor was removed from the refiner at a minimum temperature of 40.6° C.

The mixture was cooled to 34.4° C.

A 1 wt. % cocoa silk was incorporated and stirred until fully melted.

The cocoa silk tempering method provided a stable crystalline structure, ensuring a smooth texture and glossy finish.

### Final Steps

The tempered chocolate was poured into polycarbonate molds and allowed to solidify at room temperature for 24 hours.

The finished chocolate bars were demolded and analyzed for texture and appearance.

1. A method of producing chocolate, chocolate-like products, food products, or food materials, based on in vitro cacao plant cell biomass, comprising the steps of:
a) culturing an in vitro cacao plant cell biomass in a cell culture medium, optionally with at least one microorganism and/or at least one enzyme, wherein the biomass comprises somatic embryos from Theobroma cacao L. explant material;
b) separating the plant cell biomass from the cell culture medium;
c) optionally drying the biomass from step b) at least partially to obtain an at least partially dried biomass;
d) optionally roasting the biomass from step b) or at least partially dried biomass from step c) to obtain a roasted biomass;
e) optionally grinding the biomass from step b), the at least partially dried biomass from step c) or the roasted biomass from step d) to obtain a ground material;
f) optionally conching the ground material from step e) to obtain a conched material; and
g) optionally tempering the ground material from step e) or the conched material from step f).

2. The method of claim 1, wherein step a) consists of co-culturing the cacao plant cell biomass with at least one microorganism, wherein the microorganism preferably-comprises at least one of yeast, molds, bacteria, lactic acid bacteria, acetic acid bacteria or other fungi used for enzyme production in food.

3. The method of claim 1, wherein step a) consists of incubating the biomass in a liquid medium comprising at least one microorganism and/or at least one enzyme.

4. The method of claim 1, wherein step a) comprises the addition of at least one enzyme, wherein the enzyme preferably comprises at least one of a protease, a glycosidase, or a lipase.

5. The method of claim 1, further comprising the addition of at least one of a reducing sugar, an amino acid or a polyphenolic compound, prior to step d).

6. The method of claim 1, further comprising the addition of at least one volatile compound, prior to step d), wherein the compound preferably comprises at least one of linalool, furaneol or a methylpyrazine.

7. The method of claim 1, wherein the microorganism produces enzymes effective for the development of taste and/or aroma.

8. The method of claim 1, wherein step c) is carried out using hot-air or heat drying, vacuum drying, drum drying, rotary drying, freeze-drying, microwave drying, or a combination of these methods.

9. The method of claim 1, further comprising a step c′) of exposing the at least partially dried biomass of step c) to conditions effective to develop taste and/or aroma.

10. The method of claim 9, wherein the conditions effective to develop taste and/or aroma comprise at least one of controlled oxidative conditions, temperature, or humidity conditions effective to develop taste and/or aroma.

11. The method of claim 1, wherein step d) is carried out using drum roasting, air roasting, tangential roasting, packed bed roasting, continuous roasting, batch roasting, infrared roasting, continuous belt roasting, centrifugal roasting, or a combination of these methods.

12. The method of claim 1, wherein step d) is carried out in an oven at temperatures ranging between about 100 and about 190° C. for about 1 minute to about 30 minutes.

13. The method of claim 1, wherein step e) is carried out using stone grinding, ball milling, roller refining, or a combination of these methods.

14. The method of claim 1, wherein step f) is carried out using long-duration conching, short-duration conching, high-shear conching, low-shear conching, or a combination of these methods.

15. The method of claim 1, wherein step g) is carried out using tabling, seeding, machine tempering, or a combination of these methods.

16. The method of claim 1, further comprising a step d′) of adding at least one other component to the roasted biomass of step d), wherein the other component preferably comprises at least one of carbohydrates, sweeteners, vegetable fats, structuring agents, milk solids, alternative milk solids, emulsifiers, aroma, prebiotic fibers, or cocoa pulp.

17. The method of claim 1, wherein step a) is at least partially carried out in a bioreactor, preferably in a stir tank reactor, a temporary immersion system (TIS), an air-lift bioreactor, an air-culture bioreactor, or a mist or fog bioreactor.

18. The method of claim 1, wherein the in vitro cacao plant cell biomass of step a) comprises somatic embryos from Theobroma cacao L. explant material produced through at least three successive cycles of direct and/or indirect somatic embryogenesis.

19. The method of claim 18, wherein each cycle in step a) comprises generating embryos on a first medium and developing such somatic embryos on a second medium, wherein the first medium comprises at least one plant growth regulator (PGR) or hormone and wherein the second medium comprises no PGRs and no hormones, or an amount of PGRs or hormones which is less than 1 wt. %, less than 0.5 wt. %, less than 0.1 wt. % or even less than 0.01 wt. %, based on the total weight of the medium, wherein the first and second media may independently be identical or different for each cycle.

20. The method of claim 1, wherein the in vitro cacao plant cell biomass of step a) has a lipid content of at least 8 wt. % based on the total weight of the dry biomass.

21. (canceled)

22. Chocolate, chocolate-like products, food products or food materials, comprising somatic embryos (SE) from Theobroma cacao L. explant material produced through at least three successive cycles of direct and/or indirect somatic embryogenesis.

23. The products or materials of claim 22, being cocoa powder, cocoa butter or chocolate.

24. The products or materials of claim 22, characterized in that their content in Cd and/or Pb is less than 10 ppb, less than 5 ppb, less than 1 ppb or even less than 0.5 ppb, based on the total weight of the product or material.
